(19) United States
(12) Patent Application Publication
(10) Pub. No.: US 2018/0000378 A1
(43) Pub. Date: Jan. 4, 2018

(54) METHOD AND APPARATUS FOR RECORDING RESPIRATORY RATE

(71) Applicant: GENERAL ELECTRIC COMPANY, Schenectady, NY (US)

(72) Inventors: Hanna Viertio-Oja, Helsinki (FI); Panu Takala, Helsinki (FI)

(21) Appl. No.: 15/638,622

(22) Filed: Jun. 30, 2017

(30) Foreign Application Priority Data
Jun. 30, 2016 (GB) 1611345.8

(51) Int. Cl. A61B 5/08; A61B 5/087; A61B 5/113; A61B 5/083

(52) U.S. Cl. CPC A61B 5/0803; A61B 5/1135; A61B 5/0871; A61B 5/083

(57) ABSTRACT

Disclosed is an apparatus for recording respiratory rate of a subject. The apparatus comprises a first component to be arranged on the subject and away from nostrils of the subject, the first component comprises a battery, optionally electronics for the sensor, and electronics for transmitting respiration rate data; and a second component for being arranged in an area of the nose of the subject, the second component comprising at least one sensor for recording respiratory rate. Disclosed is also a method for recording respiratory rate of a subject with such an apparatus. The method comprises arranging the second component in an area of the nose of the subject; arranging the first component on the subject and no further than 30 centimeters from the second component; recording respiratory rate data with the second component, and sending the respiratory rate data to the first component; and sending the respiratory rate data from the first component to a monitor or a hub.

### TECHNICAL FIELD

The present disclosure relates to respiration monitoring. More particularly, the present disclosure relates to a method and an apparatus for recording respiratory rate of a subject.

### BACKGROUND

Breathing, which in organisms with lungs is called ventilation and includes inhalation and exhalation, is a part of respiration. Respiration rate is measured normally when a subject is at rest and involves counting the number of breaths for one minute by counting how many times the chest rises. One type of sensor measures respiration rate by sensing the airflow of the breath of the subject.

Recently attention has been given to respiration monitoring and monitors and sensors therefore. Sensors of today are often not comfortable or accurate. External airflow often influences these sensors.

### SUMMARY OF THE INVENTION

The present disclosure is directed to a method and an apparatus for recording respiratory rate of a subject. This can be achieved by the features as defined by the independent claims. Further enhancements are characterized in the dependent claims.

According to one embodiment, the present disclosure is directed to an apparatus for recording respiratory rate of a subject. The apparatus comprises a first component and a second component. The first component is configured to be arranged on the subject and away from nostrils of the subject, the first component comprises a battery, optionally electronics for the sensor, and electronics for transmitting respiration rate data. The second component is configured to be arranged in an area of the nose of the subject, the second component comprising at least one sensor for recording respiratory rate.

In one embodiment, the first component is configured to be arranged 30 centimeters or less from the second component. In one embodiment, the second component and the first component are connected to each other wirelessly or by a wire.

In one embodiment, the first and/or second component comprises an arrangement for opening up one or more nostrils of the nose. The first component may further comprise electronics and the arrangement for opening up one or more nostrils of the nose. The arrangement for opening up one or more nostrils of the nose may further comprise a tape, and the electronics may be attached to the nose by the tape. The arrangement for opening up one or more nostrils of the nose may comprise one or more arc-shaped members, and the at least one sensor may be attached to the one or more arc-shaped members. Preferably, the second component may comprise the arrangement for opening up one or more nostrils of the nose.

In one embodiment, the second component is configured to be arranged at least partly within the nose. In one embodiment, the first component is an adhesive strip configured to be arranged on the subject. In one embodiment, the first component may be configured to be arranged on, or around, the ear or nose or neck of the subject.

In one embodiment, the at least one sensor is a sensor of the following group: airflow sensor, humidity sensor, thermistor, oxygen sensor, carbon dioxide sensor, nitrogen sensor, pressure sensor, direction of airflow sensor, pulse oximeter, and SpO2 sensor.

In one embodiment, the second component may be ring shaped and comprises the at least one sensor inside the nose and a battery and/or a transmitter outside the nose. In one embodiment, a soft clip or a magnet may be configured to hold the second component in place at least partly within the nose. In one embodiment, the second component may comprise one sensor for each nostril of the nose.

In one embodiment, the battery for the first component, and/or second component, is in the form of a sticker for attachment to the skin of the subject. In one embodiment, the battery and transmitter of the first component is in the form of a tattoo for attachment to the skin of the subject. In one embodiment, the first component further comprises a pulse oximeter, or a SpO2 sensor. In one embodiment, the second component comprises a further sensor for a mouth of the subject, and/or at least one battery for the at least one sensor.

At least one embodiment discloses a method for recording respiratory rate of a subject with an apparatus according to any one of the preceding embodiments or other embodiments in this disclosure. The method comprises arranging the second component in an area of the nose of the subject; and arranging the first component on the subject and no further than 30 centimeters from the second component. These two steps may be taken in any order. The method comprises further recording respiratory rate data with the second component, and sending the respiratory rate data to the first component; and sending the respiratory rate data from the first component to a monitor or a hub.

In at least one embodiment, the second component is removable from the nose, and when removed the second component is still connected to the first component, and the first component sends a status to the monitor or the hub that the second component is not in place. In one embodiment, the first component sends a status to the monitor or the hub that the second component and the first component are not arranged on the subject, when both the second and the first component are removed from the subject.

At least one embodiment disclosed herein provides an apparatus for recording respiratory rate of a subject and a method for the same. At least one embodiment disclosed herein avoids external airflows influencing the recording of respiratory rate. At least one embodiment disclosed herein provides an apparatus that is held firmly in place. At least one embodiment disclosed herein provides an apparatus that is easy to place on a subject, is reliable and produces repeatable results, and a method providing the same. At least one embodiment disclosed herein provides a solution that is comfortable to wear, looks nice, is acceptable by a subject and fulfils social aspects. At least one embodiment disclosed herein provides a solution that is inexpensive and easy to realize in reality. At least one of the embodiments disclosed herein provides one or more solutions to the problems and disadvantages with the background art. At least one embodiment avoids sensors being swallowed. At least one embodiment provides improved patient safety and/or improved quality of respiration monitoring.

Other technical advantages of the present disclosure will be readily apparent to one skilled in the art from the following description and claims. Various embodiments of the present application obtain only a subset of the advantages set forth. No one advantage is critical to the embodiments. Any herein disclosed embodiment may be technically combined with any other disclosed embodiment or embodiments.

### BRIEF DESCRIPTION OF THE DRAWINGS

The accompanying drawings illustrate presently exemplary embodiments of the disclosure and serve to explain, by way of example, the principles of this disclosure.

FIG. 1 is a diagrammatic illustration of an apparatus according to an exemplary embodiment of the disclosure;

FIG. 2 is a diagrammatic illustration of an apparatus according to an exemplary embodiment of the disclosure; and

FIG. 3 shows a flow chart of a method according to an exemplary embodiment of the disclosure.

### DETAILED DESCRIPTION

At least one embodiment relates to a method and an apparatus for recording respiratory rate of a subject obtaining a reliable measurement of respiration. A sensor to measure one or more of flow, temperature, humidity, CO2, etc. may be placed close to, or in, the patient's airways such as the nose and/or mouth. There is a risk that most patients would dislike having a sensor device including sensing element, power source, and transmitter on their nose or face. To solve this problem, a measurement of respiration is segmented into two components, or more. The part attached on the nose may be miniaturized and designed to be acceptable and comfortable, for example as jewelry, such as for example a nose ring.

At least one embodiment relates to a method and an apparatus for recording respiratory rate of a subject. Embodiments may comprise respiratory rate measurement with one or more components that keep the subject's nostrils open to facilitate effortless breathing. Such a component may be, for example, an anti-snoring strip keeping the nostrils open and holding a power source as well as transmitter electronics of the measurement. Another example of such a component may be tubes placed in the nostrils keeping the nostrils open and holding a sensor, a thermistor for example, in an optimal position for detecting air flow caused by breathing.

In at least one embodiment relating to a method and an apparatus for recording respiratory rate of a subject, the apparatus may comprise two components. One component attached on the face, around or on the nose, may be miniaturized and designed to be nice looking, acceptable and comfortable. This may look like a nose ring, with a sensor part inside the nostril and a transmitter and a small power source outside the nose integrated in the ring. To minimize the power source, the other component may be arranged on the ear, so that the distance to transmit information between the two components would be minimized in order to minimize power source required in the nose part. Preferably such a distance would be 30 cm or less. Alternatively, or in combination, there may be a wired connection from the nose to the ear, with the possibility to remove the sensor from the nose when it is not needed and let it hang from the ear, for example when the patient goes to a cafe. Various other sensors may be integrated in these exemplary embodiments. SpO2 may be measured from the nose ring or from the ear. Core temperature may be measured from the ear as well. A soft clip or a magnet may be used to hold the nose sensor in place. An accelerator may be included in one part to sense movement of the subject. Embodiments disclosed herein may comprise a first component, which may comprise a hearing-aid sized housing, housing a processor, a transmitter and a power supply, on the patient's ear with possibly additional sensors. Embodiments disclosed herein may comprise a second component in an area of, around, the nose, the first component being comfortable and acceptable for the wearer and onlookers.

In at least one embodiment, the second component may be located close to the subject's airways, nose and/or mouth. The second component may be arranged in an area around the nose. This may be on the skin of the nose and/or inside the nostrils of the nose. Such an arrangement would result in a reliable respiration measurement, and gives the subject comfort and acceptability together with a reliable measurement. This would result in patients not objecting to wear such an apparatus for recording respiratory rate on their face.

In at least one embodiment, the second component may assist a subject to breathe easier. The second component may comprise one or more strips placed over the nose and/or a tubing that keeps the nostrils open by expanding them from their inside. A sensor for measuring a subject's respiratory rate may comprise recording variation of temperature or humidity of the air flowing in and out of the subject's nose. A tubing, arc-shaped members, or any other such arrangement for keeping the nostrils open, may allow for easy and accurate placement of the sensor in the air stream giving a high quality of the signal.

One embodiment may comprise an adhesive strip that expands the nostrils from the outside. In addition, in a further embodiment, the adhesive strip may hold one or more of a battery, sensor electronics, and transmitter electronics of a respiration rate recording system. In addition or as an alternative, in a further embodiment, tubes, or any other arrangement for keeping the nostrils open, placed inside the nostrils keep the airways open and may hold a sensor element, thermistor for example, accurately in the air stream. In a further embodiment, the adhesive strip may serve as a risk mitigation mechanism that prevents the patient from swallowing the nose tubes and the sensor. One or more embodiments disclosed herein provides for a robust respiration rate measurement with a light-weight and reasonably unobtrusive configuration. In at least one embodiment, the apparatus may reduce resistance of the airflow in the subject's nose, thus providing a better signal for the respiratory rate measurement, and may measure accurately the respiratory rate because the sensors are kept firmly in place in the nostrils.

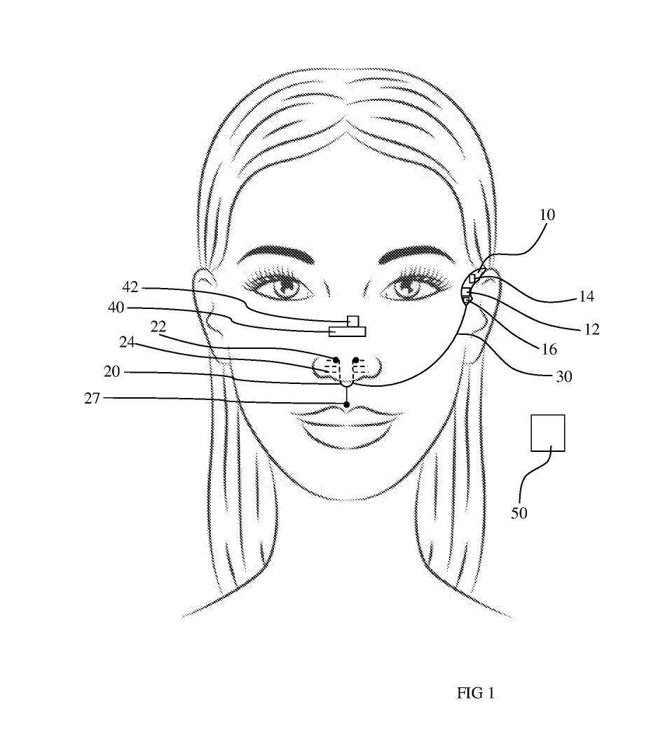

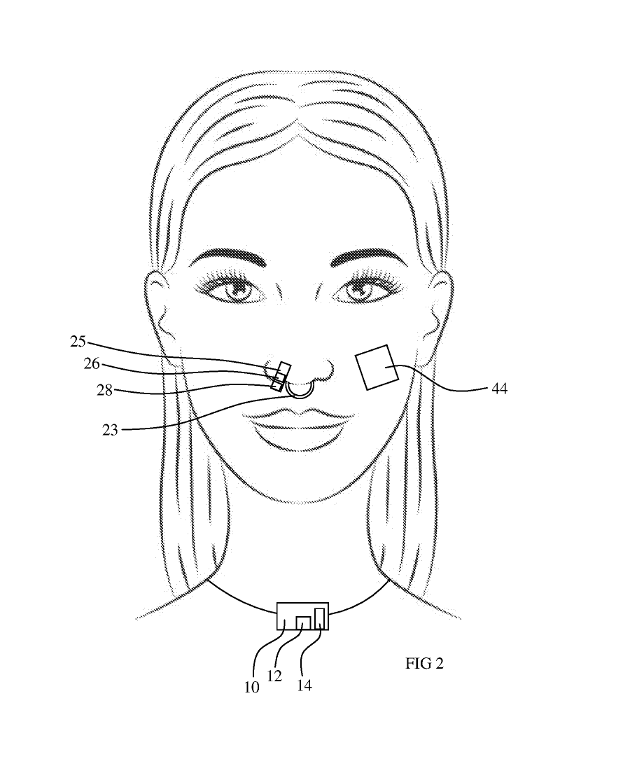

Turning to FIGS. 1 and 2, several exemplary embodiments are schematically illustrated on a subjects face. For example, one embodiment illustrates an apparatus for recording respiratory rate of a subject comprising a first component 10 and a second component 20. The first component 10 may be configured to be arranged on the subject and away from the nostrils of the subject. The first component 10 may comprise a battery 12, optionally electronics 14 for the sensor, and electronics 14 for transmitting respiration rate data. The second component 20 may be configured to be arranged in an area around the nose of the subject. The second component 20 may comprise at least one sensor 22 for recording respiratory rate. According to at least one further embodiment, the second component 20 may further comprise a battery 26 and/or electronics 28, such as a transmitter 28.

The electronics 14 for the sensor may be a microprocessor or any other electronics for converting the sensed signals into data. In some embodiments the electronics 14 may be present; in other embodiments the electronics 14 may not be present or necessary. In at least one embodiment the electronics 14 for the sensor are not necessary because the sensor 22 outputs data that can be transmitted directly to the first component. The electronics 14 for transmitting respiration data may be a transmitter communicating, for example, to the second component 20 or directly to a monitor or hub of a medical network. Such a transmitter may communicate via a wireless protocol such as Bluetooth, WI-FI, a medical body area network, near field communication, etc.

The transmitter 28 may transmit data sensed by the sensor 22, for example to the first component 10 or directly to a monitor or hub of a medical network. The transmitter 28 may communicate via a wireless protocol such as Bluetooth, WI-FI, a medical body area network, near field communication, etc. The transmitter 28 may be used when the second component 20 is wireless connected to the first component 10, and the transmitter 28 may be omitted when the first and second components are connected by a wire.

In at least one embodiment herein, the first and second components 10, 20 are configured to be located at different locations on the subject. The first component 10 comprising larger and/or heavier parts, such as the battery 12 and electronics 14 may be arranged away from the nose or away from the nostrils, but still on the subject, preferably on a location on the head and/or neck area of the subject and/or the face of the subject. For example, the first component 10 may be configured to be arranged on and/or around the ear of the subject, around the neck of the subject, around/in the hair, on the cheek, on the forehead, on the nose, or around the head, or one or more combinations hereof. The second component 20 comprises smaller and generally lighter parts, compared with the first component 10, such as at least one sensor 22. The second component 20 may be configured to be arranged in an area of the nose of the subject. Such an area around the nose may comprise locations on the exterior of the nose and/or locations inside the nose. The second component 20 may be configured to be arranged in one or both nostrils. The second component 20 may be partly within a nostril and partly outside the nostril, such as for example a clamp or ring on the nose. While the two components 10, 20 are arranged apart from each other, they are still so close to each other that the can operatively connect to each other, for example by wire or wirelessly. In embodiments herein, the first component 10 is arranged 30 centimeters or less from the second component 20. In embodiments herein, the first component 10 is arranged 25 centimeters or less from the second component 20. In embodiments herein, the first component 10 is arranged 20 centimeters or less from the second component 20.

By separating the apparatus for recording respiratory rate into at least two components, the apparatus is comfortable to carry and wear by the subject. Each component may be made to fit and/or to look nice so that the subject even can experience a “jewelry” effect. As such the apparatus will be socially acceptable. The components are easy to place and their positioning and/or result will be reliable and repeatable. The components can be held firmly in place on the subject. External air flows may be avoided given that the second component can be arranged so close to, or even inside, the nose.

In at least one embodiment herein, the second component 20 and the first component 10 are connected to each other wirelessly or by a wire 30. The wire 30 may be an electric wire for electric power and/or for electric signals, such as electric signal to or from the sensor 22. The wire 30 may be a string, an elastic band, or similar for a physical connection. By using a wire 30 between the two components 10, 20 it can be assured that the second component is not lost or swallowed by the subject. In this way the apparatus can comply with safety regulations.

In at least one embodiment herein, the first and/or second component comprises an arrangement for opening up one or more nostrils of the nose. The arrangement can be done in several different ways, for example tubing, rings, or other arc-shaped members, and further exemplary embodiments are given herein.

In at least one embodiment, the first component 10 further comprises electronics and the arrangement for opening up one or more nostrils of the nose. The arrangement for opening up one or more nostrils of the nose may be a tape 40, and the electronics 42 may be attached to the nose by the tape 40. The tape 40 may be a sports man tape that is attached on the skin on the outside of the nose to open up the air flow through the nose.

In at least one embodiment, the arrangement for opening up one or more nostrils of the nose may comprise one or more arc-shaped members 24, and the at least one sensor 22 may be attached to the one or more arc-shaped members 24. In another embodiment, the second component may comprise the arrangement for opening up one or more nostrils of the nose. In embodiments, both the first and second components may comprise an arrangement for opening up one or more nostrils of the nose, for example the one or more arc-shaped members 24 may be used in combination with the tape 40. In this way the tape 40 may hold the electronics 42 and opens up the nostrils. The one or more arc-shaped members 24 may hold the sensor and also opens up the nostrils. Additional components may be on a third place on the subject, for example behind the ear. Hereby savings in power consumption may be made by sending the signal from the sensor to the tape 40 on the nose and from the tape 40 subsequently to the additional component behind the ear or directly to a monitor or hub 50 in embodiments with no additional component. The additional, third, component may be configured to be arranged on and/or around the ear of the subject, around the neck of the subject, around/in the hair, on the cheek, on the forehead, or around the head, or one or more combinations hereof.

In at least one embodiment, the second component 20 is configured to be arranged at least partly within the nose. For example, the second component 20 may be in the form of a clip, or ring, or holder that is partly within the nose, for example partly within one or more nostrils. A part of the second component 20 may be configured to be arranged on the outside of the nose and a part of the second component 20 may be configured to be arranged within one or more nostrils.

In at least one embodiment, the first component 10 may be an adhesive strip configured to be arranged on the subject. In one embodiment, the first component 10 is configured to be arranged on, or around, the ear or nose or neck of the subject. In a further exemplary embodiment, the first component may be configured as a hair band, or head phones, or cap, or similar.

In at least one embodiment, the at least one sensor 22 is a sensor of the following group: airflow sensor, humidity sensor, thermistor, oxygen sensor, carbon dioxide sensor, nitrogen sensor, pressure sensor, direction of airflow sensor, pulse oximeter, and SpO2 sensor. The at least one sensor 22 may be arranged on the second component 20, but an additional sensor may be arranged on the first component 10 and/or the second component 20. In one embodiment, there may be two sensors 22, one in each nostril of the subject.

In at least one embodiment, the second component may be ring shaped and comprises the at least one sensor 22 inside the nose and a battery 26 and/or electronics 28, such as for example a transmitter 28, outside the nose. For example, a ring 23, as the second component 20, may be going from one nostril to the other nostril. For example, a ring 23, as the second component 20, may be going through only one single nostril. Embodiments of the ring 23 may not be a closed ring but a ring with a small opening so that the ring 23 can be clamped onto the nose. The at least one sensor 22 is configured, on the ring 23, to be inside the nose, the nostril, so that an accurate recording of respiratory rate may take place. In one embodiment, the ring 23 may function as an antenna. This may be particularly useful for making the second component small, light, and social acceptable to wear.

In at least one embodiment, a clip 25 or a magnet 25 holds the second component in place at least partly within the nose. Both the clip and the magnet are illustrated with the same reference number 25. Preferably the clip 25 is a soft clip 25. In one embodiment, a part of the clip 25 or magnet 25 may be inside one nostril and another part of the clip 25 and magnet 25 may be on the outside of the nose, or inside the other nostril. In one embodiment, the clip or magnet may function as an antenna. This may be particularly useful for making the second component small, light, and social acceptable to wear.

In at least one embodiment, the second component 20 may comprise one sensor 22 for each nostril of the nose. For example, the arrangement 24 for opening up one or more nostrils of the nose, for example the arc-shaped member 24, may comprise one sensor 22 for each nostril. In this way a very accurate recording of respiratory rate may take place.

According to at least one embodiment, the battery 12 for the first component 10, and/or the battery 26 for the second component 20, is in the form of a sticker 44 for attachment to the skin of the subject. According to at least one embodiment, the battery 12 and electronics 14, for example transmitter 14, of the first component 10 is in the form of a tattoo 44 for attachment to the skin of the subject. This may also be used for the battery 26 and the electronics 28 of the second component 20. The sticker and the tattoo are illustrated using the same reference number 44. Using a sticker and/or tattoo may result in a comfortable, socially acceptable, and light battery to carry on the skin by the user. The sticker may be in the form of a traditional sticker with an adhesive on one side for attaching the sticker to the skin of the subject. The tattoo may not be permanent, but could be applied for several days to the skin of the subject. The tattoo may have a decoration that appeals to the wearer. The tattoo may be applied to the skin of the subject in any form. The sticker, or tattoo, may be incorporated as, or function as, or be part of, the wire 30.

In at least one embodiment, the first component 10 further comprises a pulse oximeter, or a SpO2 sensor. Such a sensor is illustrated by reference number 16 in FIG. 1. The first component 10 may have, in addition to the one or more sensors of the second component 20, a further sensor 16 so that data in addition to respiratory data may be collected from the subject. The sensor 16 of the first component 10 may be, for example, clipped on to the earlobe of the subject.

In at least one embodiment, the second component 20 comprises a further sensor 27 for a mouth of the subject. A mouth sensor 27 may be used in addition, or separately from the at least one sensor 22 of the second component 20. The sensor 27 for the mouth may also be for respiratory rate, or for other data. The second component 20 may comprise a connectable third component comprising the sensor 27. In at least one embodiment, the third component is connected to the second component 20 and extends from the nose area, or from the nostrils, to the mouth area of the subject and comprises the sensor 27 for the mouth.

In at least one embodiment, the first component 10 and the second component 20 are configured to be arranged in one or more nostrils. In such a case both components are so small that they fit inside one or both nostrils of a subject. The first component and the second component could be integrated with each other. In one embodiment, the battery and electronics for transmitting respiration rate data are configured to be outside the one or more nostrils. In this way it is possible to carry an antenna and a larger battery with the sensor. The antenna may be comprised by the electronics for transmitting respiration rate data of the first or second component and may have a better range of transmission because it is outside the nostril.

In at least one embodiment, the second component 20 comprises at least one battery 26 for the at least one sensor. In one exemplary embodiment, the first component 10 and the second component 20 may be connected wirelessly and the battery 26 may then be small because it only needs to power data to be sent between the first and second components.

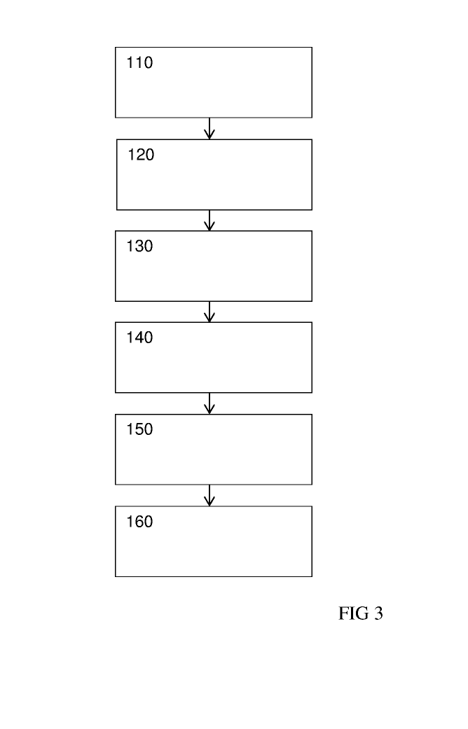

According to at least one embodiment, a method for recording respiratory rate of a subject with the apparatus according to any one of the embodiments disclosed herein is illustrated by the flow chart of FIG. 3. FIG. 3 shows a flow chart of a method for recording respiratory rate of a subject. The first two steps can be taken in any order. A first step, illustrated as 110 in FIG. 3, comprises arranging the second component 20 in an area of and/or, around, the nose of the subject. A second step, illustrated as 120 in FIG. 3, comprises arranging the first component 10 on the subject and no further than 30 centimeters from the second component. The first component is arranged on the subject and preferably away from nostrils of the subject. The second step 120 may be taken before or at the same time as the first step 110. A third step, illustrated as 130 in FIG. 3, comprises recording respiratory rate data with the second component, and sending the respiratory rate data to the first component. A fourth step, illustrated as 140 in FIG. 3, comprises sending the respiratory rate data from the first component to a monitor 50 or a hub 50. Both the hub and the monitor are illustrated with reference number 50 in FIG. 3.

By separating the apparatus for recording respiratory rate into at least two components, the apparatus is comfortable to wear for the subject. The second component needs only to send data to the first component, not further away and can therefore be made smaller and lighter. Each component may be made to fit and/or to look nice so that the subject even can experience a “jewelry” effect. As such the apparatus will be socially acceptable. The components become easy to place and their result and/or placement will be reliable and repeatable. The components can be held firmly in place on the subject. External air flows may be avoided given that the second component can be arranged so close to the nose.

In at least one embodiment, the second component 20 is removable from the nose. A further method step, illustrated as 150 in FIG. 3, comprises that when the second component 20 is removed, the second component is still connected to the first component 10, and the first component 10 sends a status to the monitor 50 or the hub 50 that the second component is not in place. The two components 10, 20 may be still connected to each other when a wire 30 connects them. The connection may be made by a string, an elastic band, or similar.

In at least one embodiment, the first component 10 sends a status to the monitor 50 or the hub 50 that the second component 20 and the first component 10 are not arranged on the subject, when both the second 20 and the first component 10 are removed from the subject. The status sent may be a simple signal. In this way the loss or removal of the two components 10, 20 may be noticed and/or recording respiratory rate may be stopped.

In at least one embodiment, the method and apparatus records respiration rate. At least on embodiments provides for a safe system, preventing swallowing of components. The separation of the two components allow weight, size, and power, to be distributed improving appearance, comfort, and capability of sending respiration rate data to a hub or monitor. Improved quality of respiration rate measurement increases customer satisfaction and promotes sales. The disclosed advantages and technical advantages above apply for the embodiments of the methods as well as the embodiments of the apparatus.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. An apparatus for recording respiratory rate of a subject, comprising
a first component configured to be arranged on the subject and away from nostrils of the subject, the first component comprises a battery, and electronics for transmitting respiration rate data; and
a second component configured to be arranged in an area of the nose of the subject, the second component comprising at least one sensor for recording respiratory rate.

2. The apparatus according to claim 1, wherein the first component is configured to be arranged 30 centimeters or less from the second component.

3. The apparatus according to claim 1, wherein the second component and the first component are connected to each other wirelessly or by a wire.

4. The apparatus according to claim 1, wherein the first or second component, or both the first and second components, comprise an arrangement for opening up one or more nostrils of the nose.

5. The apparatus according to claim 4, wherein
the first component further comprises the arrangement for opening up one or more nostrils of the nose; and
the arrangement for opening up one or more nostrils of the nose comprises a tape, and the electronics for transmitting respiration rate data being attached to the nose by the tape.

6. The apparatus according to claim 4, wherein
the arrangement for opening up one or more nostrils of the nose comprises one or more arc-shaped members, and the at least one sensor is attached to the one or more arc-shaped members; and
wherein, preferably, the second component comprises the arrangement for opening up one or more nostrils of the nose.

7. The apparatus according to claim 1, wherein the second component is configured to be arranged at least partly within the nose.

8. The apparatus according to claim 1, wherein the first component is an adhesive strip configured to be arranged on the subject, and/or wherein the first component is configured to be arranged on, or around, the ear or nose or neck of the subject.

9. The apparatus according to claim 1, wherein the at least one sensor is a sensor of the following group: airflow sensor, humidity sensor, thermistor, oxygen sensor, carbon dioxide sensor, nitrogen sensor, pressure sensor, direction of airflow sensor, pulse oximeter, and SpO2 sensor.

10. The apparatus according to claim 1, wherein the second component is ring shaped and comprises the at least one sensor inside the nose and a battery and/or a transmitter outside the nose.

11. The apparatus according to claim 1, wherein a soft clip or a magnet is configured to hold the second component in place at least partly within the nose.

12. The apparatus according to claim 1, wherein the second component comprises one sensor for each nostril of the nose.

13. The apparatus according to claim 1, wherein the battery for the first component, and/or second component, is in the form of a sticker for attachment to the skin of the subject.

14. The apparatus according to claim 1, wherein the battery and transmitter of the first component is in the form of a tattoo for attachment to the skin of the subject.

15. The apparatus according to claim 1, wherein the first component further comprises a pulse oximeter, or a SpO2 sensor.

16. The apparatus according to claim 1, wherein the second component comprises a further sensor for a mouth of the subject, and/or at least one battery for the at least one sensor.

17. The apparatus according to claim 1, wherein the first and second components are configured to be arranged at least partially in one or more nostrils, optionally the battery and electronics for transmitting respiration rate data of the first component are configured to be outside one or more nostrils.

18. A method for recording respiratory rate of a subject with an apparatus according to any one of the preceding claims, the method comprising:
providing a first component having a battery and electronics for transmitting respiration rate data;
providing a second component having at least one sensor for recording respiratory rate;
arranging the second component in an area of the nose of the subject;
arranging the first component on the subject and no further than 30 centimeters from the second component;
recording respiratory rate data with the second component, and sending the respiratory rate data to the first component; and
sending the respiratory rate data from the first component to a monitor or a hub.

19. The method of claim 18, wherein the second component is removable from the nose, and when removed the second component is still connected to the first component, and the first component sends a status to the monitor or the hub that the second component is not in place.

20. The method of claim 18, wherein the first component sends a status to the monitor or the hub that the second component and the first component are not arranged on the subject, when both the second and the first component are removed from the subject.
